(19) United States
(12) Patent Application Publication
(10) Pub. No.: US 2018/0161036 A1
(43) Pub. Date: Jun. 14, 2018

(54) ENDOSCOPIC CLIP

(71) Applicant: ENDOGEAR LLC, Seattle, WA (US)

(72) Inventors: Benjamin Merrifield, Seattle, WA (US); Adam Smith, Seattle, WA (US); Drew Schembre, Seattle, WA (US)

(73) Assignee: Endogear LLC, Seattle, WA (US)

(21) Appl. No.: 15/109,622

(22) PCT Filed: Mar. 4, 2016
(86) PCT No.: PCT/US2016/021046

§ 371 Date: Jul. 1, 2016

Related U.S. Application Data
(60) Provisional application No. 62/128,311, filed on Mar. 4, 2015.

(51) Int. Cl. A61B 17/122; A61B 17/08; A61B 17/128

(52) U.S. Cl. CPC A61B 17/122; A61B 2017/0034; A61B 17/1285; A61B 17/083

(57) ABSTRACT

The present invention relates to clips and, specifically, to endoscopic clips. Clips of the present invention comprise a rail, a proximal prong, and a distal prong. Clips of the present invention comprise multiple sequentially deployed folding prongs, which may deploy in a substantially perpendicular manner by rotating from a folded position flush with a rail to about a 90° angle relative to the rail. The proximal prong may be both rotatable and longitudinally slidable relative to the rail. The distal prong may be rotatable relative to the rail. Folded clips of the present invention may be delivered to a target site within a sheath. The sheath may be retracted from the folded clips to permit deployment and the sheath end may be subsequently used to push the longitudinally slidable proximal prong towards the distal prong for clamping. The clip is releasable, and may be released from a latch wire.

### CROSS-REFERENCE TO RELATED APPLICATIONS

This application claims priority to U.S. Provisional Patent Application Ser. No. 62/128,311, titled ZIP CLIP ENDOSCOPIC HEMOSTASIS DEVICE, filed on Mar. 4, 2015 and PCT Patent Application Serial No. PCT/US2016/021046, titled ENDOSCOPIC CLIP, filed on Mar. 4, 2016.

### FIELD OF THE INVENTION

The present invention relates to clip devices and, specifically, endoscopically delivered clip devices that may be used to stop gastrointestinal bleeding, mark locations, manipulate tissue, and close perforations and mucosal defects. While this invention is described in terms of endoscopic clip devices and systems in detail here, due to its slim delivery profile, unique and advantageous orientation, folding lockable clamp arms, and suitability for remote highly manipulable actuation to provide clamping or closure in otherwise difficult to access locations, the present clip device is contemplated to have several applications beyond endoscopic use. Additional applications may take advantage of some or all of the principal features of the present invention and also be adapted for larger functional purposes such as, for example, use in construction and repair, waste securement and/or retrieval, maneuverability and handling of toxic substances, access to items crowded and or high shelves, or any application where remote closure, clamping, grabbing, or retrieval of items from otherwise difficult to access locations is desired.

### SUMMARY

The endoscopic clip device of the present invention includes several features that render it particularly well-suited for effective use in tight spaces, such as those accessible by endoscopes, and a signification improvement over conventional endoscopic clips. One, some, or all of the following features is provided by various embodiments of the present invention. Description of the many advantageous features provided by the endoscopic clip device of the present invention and the Figures depicting some of its inventive embodiments also provide information as to the functional configuration and its component parts, such that methods of its use and application may be readily understood.

First, the pre-deployment structural configuration of the endoscopic clip device includes prongs that are folded flush against or close to the surface of the rail such that it fits neatly and securely within an endoscopic sheath. This low-profile configuration provides for smooth low profile access and delivery of the clip device to a target tissue site via a flexible endoscope.

Second, the folding prongs are only deployed into a substantially perpendicular position (about 90° angle) relative to the clip rail or backbone at a location proximal to a target tissue site as the sheath covering is removed from the folded prongs located on an outer surface of the endoscopic clip. Currently available endoscopic clips emerge from the endoscope in a “V-shape,” facing forward. This is suitable for clipping targets and defects directly in front of the scope but pose more of a problem when the target is oblique to the tip of the scope. In the narrow space of the colon or small bowel, for example, most targets lie lateral to, not directly in front of the endoscope. Because the endoscopic clip emerges from a tubular sheath generally in longitudinal orientation with the surrounding or proximal target tissue site locations, deployment of the folding prongs into a substantially perpendicular configuration along this longitudinal axis will, in many instances, automatically be in a generally suitable orientation such that the deployed prongs are facing opposite to the target tissue site. This means that less manipulation and bending of the endoscopic clip device of the present invention is required to orient the clip grasping prongs to the target tissue site than is necessitated by, for example, “V-shaped” clips which are deployed in an orientation that is facing away from the target tissue site.

Third, the longitudinal folding prong configuration wherein each folding prong is located in a spaced apart location along a connecting rail provides an endoscopic clip having a greater clamping range than conventional “V-shaped” clips or clips where each prong end is attached to the same point or located closely adjacent to the other upon deployment.

Fourth, upon deployment, the folding prongs rotate one-way about 90° into a locked perpendicular position relative to a central longitudinal rail due to the rotational fit of interactive folding prong and rail components. Relative to, for example, conventional “V-shaped” clips, the flat substantially perpendicular clamping surface provided by the deployed folding prong can provide more uniform pressure to clamped tissue such that the purchase grip provided by the present inventive clip is greater than that provided by conventional “V-shaped” clips which can be more likely to slip or lose their purchase strength.

Fifth, the folding prongs of the present invention are held under tension within the endoscopic sheath which, upon removal of the sheath, results in the rotational movement of the folding prongs into a substantially perpendicular position, whereby each folding prong is located opposite to and faces the other. The tension strength included for use with the present invention is highly adaptable, and may be adjusted for use with clips having different applications and tolerances.

Sixth, the clip device of the present invention comprises a proximal actuator or moving handle attached to a latch wire associated with manipulable tension component, such as, for example, a spring. The latch wire passes from the proximal actuator or moving handle through the endoscope channel and terminates at a point within a latch plate retained within the rail of the clip mechanism located at the distal tip of the endoscopic clip device. The tension associated with the latch wire causes the rotatable movement of the folding prongs.

Seventh, the clip mechanism at the distal tip of the endoscopic clip device of the present invention is releasable. That is, each clip can be deployed, tightened on targeted tissue, and then selectively released. In a preferred embodiment, the clip that remains in place after separation from the remaining endoscopic clip device components includes a rail and two prongs. In another preferred embodiment, the clip that remains in place after separation from the remaining endoscopic clip device components includes a rail, two prongs, and a latch plate. The clip then remains in place at the targeted tissue site.

Eighth, the present invention provides for a highly adjustable manually controlled clip clamping force that is applied after deployment of the clip by pressing the end of the sheath, which can be connected to a fixed handle, against the back of the deployed proximal prong to slide the proximal prong along the rail towards the distal prong, all while holding the distal prong in place with the latch wire which is controlled via a proximal actuator or moving handle. In a preferred embodiment, the clip clamping force applied by pressing on the proximal prong using the sheath end provides for a precise directly manual variable clamping force that is conveniently controlled remotely by application of force on the sheath by the user at the hand piece. In one embodiment, the user provides a pushing force on the sheath to cause longitudinal movement of the proximal prong along the rail towards the distal prong; meanwhile the distal prong is held, or retained, in place by an opposite tension force delivered to the distal prong via the latch wire.

Ninth, upon deployment, the clamped clip is locked in place by the close frictional fit provided at the junctures between the rail and each of the close-fitting prongs. In layman's parlance, this type of frictional fit is referred to as a “dresser drawer” fit because, like a slidable dresser drawer, the deployed prongs have very little vertical or horizontal tolerance for movement.

Tenth, the present invention also provides a potentially infinitely variable clamp and release distance such as anywhere between, for example, about 10 mm and about 1 mm. Further, the multi-purpose utility of the sheath, which includes a pressing functionality along with a protective and delivery functionality, further minimizes part count and reduces points of potential device failure.

Eleventh, the clip release is separately achieved by sliding a separate tube, which is nested within the flexible shaft and coupler, towards and pressing against the snap fitted flexible latch wire catch point, such as a latch ledge. The snap fitted flexible latch wire catch point secures the latch wire to the releasable clip components (e.g., the latch plate, rail, and prongs of the releasable clip); thus, causing the snap fitted flexible latch wire catch point to disengage from the latch wire and freeing the releasable clip components from the remainder of the endoscopic clip device components. Another benefit provided by one embodiment of this clip release mechanism is that, because it has a range of motion completely nested within the rail component, it provides a smooth exit mechanism because there are no protruding components to catch on the surrounding tissue or on the delivery components of the device during release of the releasable clip components or retraction and removal of the remainder of the endoscopic clip device components from the target tissue site.

Twelfth, the clip release can be accomplished by simply removing the hold on the same latch wire and tension mechanism used to deploy the clip prongs and hold the distal prong in place which, again, by increasing the functionality of the component parts further minimizes part count and reduces points of potential device failure.

Thirteenth, users of the present invention can advantageously remotely rotate and orient the clip prongs about the endoscopic longitudinal axis by rotating and manipulating the flex cable located within the sheath using the hand controls which, in turn, provide precise rotational control without rotating the sheath within the endoscope.

In addition to the above-noted numerous attributes, the present invention beneficially provides increased effectiveness, intuitive tangential application along a longitudinal axis upon deployment, an improved ability to move and manipulate tissue, decreased procedure time, reduced medical waste, and adjustable clamping strength.

While particular embodiments of the present invention are described in detail below, it is noted that various design configurations may take advantage of one or more of the beneficial design configurations and above-noted features discovered and made possible by the present inventors.

### BRIEF DESCRIPTION OF THE FIGURES

FIGS. 1-1, 1-2, 1-3, 1-4, and 1-5 are photographs of the initial prototype of the clip concept informing the various embodiments disclosed herein and otherwise contemplated by the inventors. Upon bench top testing, this prototype proved efficacy of the proposed clamping mechanism using simulated tissue. Upon deployment, this clip prototype gathered tissue, and tighten with a desired force.

FIG. 2 shows an isometric view of the distal end of the endoscopic clip system protruding from the distal end of a generic endoscope shown for reference. The sheath has been cut back for clarity.

FIG. 3 shows an exploded view of FIG. 2.

FIG. 4 shows an isometric view of the distal end of the endoscope with the distal end of the endoscopic clip system roughly flush with the end of the endoscope.

FIG. 5 shows an isometric view of the distal end of the endoscope with the distal end of the endoscopic clip system protruding from the distal end of the endoscope with the sheath fully covering the distal end of the endoscopic clip system.

FIG. 6 shows an isometric view of the distal end of the endoscope with the distal end of the endoscopic clip system protruding from the distal end of the endoscope with the sheath fully exposing the distal prong, allowing said prong to rotate.

FIG. 7 shows an isometric view of the distal end of the endoscope with the distal end of the endoscopic clip system protruding from the distal end of the endoscope with the sheath fully exposing both the distal prong and proximal prong allowing both said prongs to rotate.

FIG. 8 shows an isometric view of the distal end of the endoscope with the distal end of the endoscopic clip system protruding from the distal end of the endoscope with the end of the sheath pushed up against the back of the proximal prong.

FIG. 9 shows an isometric view of the distal end of the endoscope with the distal end of the endoscopic clip system protruding from the distal end of the endoscope with the end of the sheath pushed up against the back of the proximal prong causing said prong to travel roughly half the distance down the rail.

FIG. 10 shows an isometric view of the distal end of the endoscope with the distal end of the endoscopic clip system protruding from the distal end of the endoscope with the end of the sheath pushed up against the back of the proximal prong causing said prong to travel completely down the rail.

FIG. 11 shows an isometric view of the distal end of the endoscope with the distal end of the endoscopic clip system protruding from the distal end of the endoscope with the end of the sheath pulled back to expose the entire rail in order to prepare for release.

FIG. 12 shows an isometric view of the distal end of the endoscope with the distal end of the endoscopic clip system protruding from the distal end of the endoscope with the end of the sheath pulled back to expose the entire rail while the coupler is fully engaged into the rail. This view also represents the release of the latch wire from the latch plate. This is obscured in this view, but is visible in FIGS. 15 and 16.

FIG. 13 shows an isometric view of the distal end of the endoscope with the distal end of the endoscopic clip system protruding from the distal end of the endoscope with the end of the sheath pulled back to expose the entire rail. This figure represents the retracting of the latch wire into the tube. This is obscured in this view, but is visible in FIGS. 17 and 18.

FIG. 14 shows the distal end of the endoscopic clip system pulled away from the endoscopic clip assembly.

FIG. 15 shows a cross sectional view of FIG. 12.

FIG. 16 shows a detail view of the distal end in FIG. 15.

FIG. 17 shows a cross sectional view of FIG. 13.

FIG. 18 shows a detail view of the distal end in FIG. 17.

FIG. 19 shows a cross sectional view of FIG. 14.

FIG. 20 shows a detail view of the distal end in FIG. 19.

FIG. 21 shows an isometric view of the rail and the distal clip in a fully stowed position.

FIG. 22 shows an isometric view of the rail and the distal clip in a partially deployed position.

FIG. 23 shows an isometric view of the rail and the distal clip in a fully deployed position.

FIG. 24 shows a side view of the proximal end of the endoscopic clip system.

### DETAILED DESCRIPTION

The following reference numbers, even if not specifically called out in each figure, are uniformly used across figures to refer to different views of the same components or features, as follows: 10 proximal prong (attached to rail 24); 12 push tab (top of proximal prong 10); 14 slide tab (top of proximal prong 10); 16 distal prong (attached to rail 24); 18 slide tab (top of distal prong 16); 20 bridge (top of distal prong 16); 22 teeth (bottom of distal prong 16); 24 rail (distal end of endoscopic clip system, until release); 26 latch slot (four locations at the bend region within rail 24); 27 rail sidewall (both vertical walls of rail 24); 28 prong slot (four locations within side walls of rail 24); 30 latch plate (snapped into rail 24); 32 latch slide tabs (two places at bottom of latch plate 30); 34 latch guide tabs (two places at top of latch plate 30); 36 latch hook (one place at bottom of latch plate 30); 38 latch flex region; 40 latch wire (concentrically aligned within tube 44); 42 latch ledge (located at the distal end of latch wire 40); 44 tube (concentrically affixed within coupler 48); 46 tube end (distal face of tube 44); 48 coupler (concentrically aligned with flex shaft 52); 50 coupler end (distal face of coupler 48); 52 flex shaft (concentrically aligned within sheath 54); 54 sheath (concentrically aligned within endoscope 58); 56 sheath end (distal face of sheath 54); 58 endoscope (contains passage for endoscopic clip system to pass through); 60 moving handle (translationally mounted to fixed handle 62); 62 fixed handle (connected to sheath 54); 64 release button (translationally mounted to moving handle 60); 66 rotation wheel (rotationally mounted within moving handle 60 and affixed to the proximal end of flex shaft 52); 68 tension spring (compressed between release button 64 and rotation wheel 66 and captured within moving handle 60).

In one embodiment of the present invention, each of these components or features comprises 316 stainless steel or a similar such material, except for 54 sheath and 56 sheath end which are made of polytetrafluoroethylene (PTFE) or a similar such material, 58 endoscope which may be made of various materials, and 60 moving handle, 62 fixed handle, 64 release button, and 66 rotation wheel which may be made of PC/ABS or a similar plastic or other material.

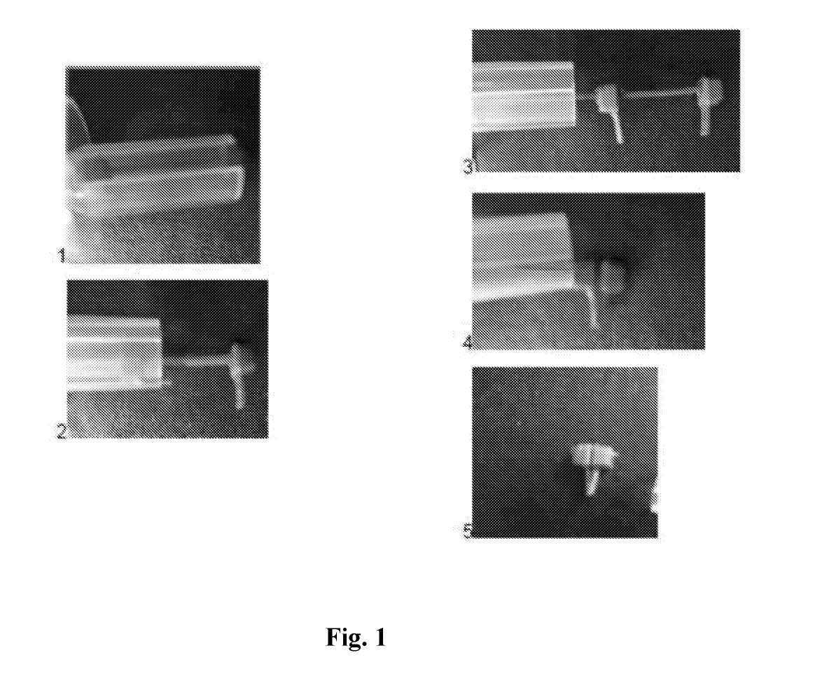

FIGS. 1-1, 1-2, 1-3, 1-4, and 1-5 are photographs of the initial prototype of the clip concept informing the various embodiments disclosed herein and otherwise contemplated by the inventors. Upon bench top testing, this prototype proved efficacy of the proposed clamping mechanism using simulated tissue. Upon deployment, this clip prototype gathered tissue, and tighten with a desired force.

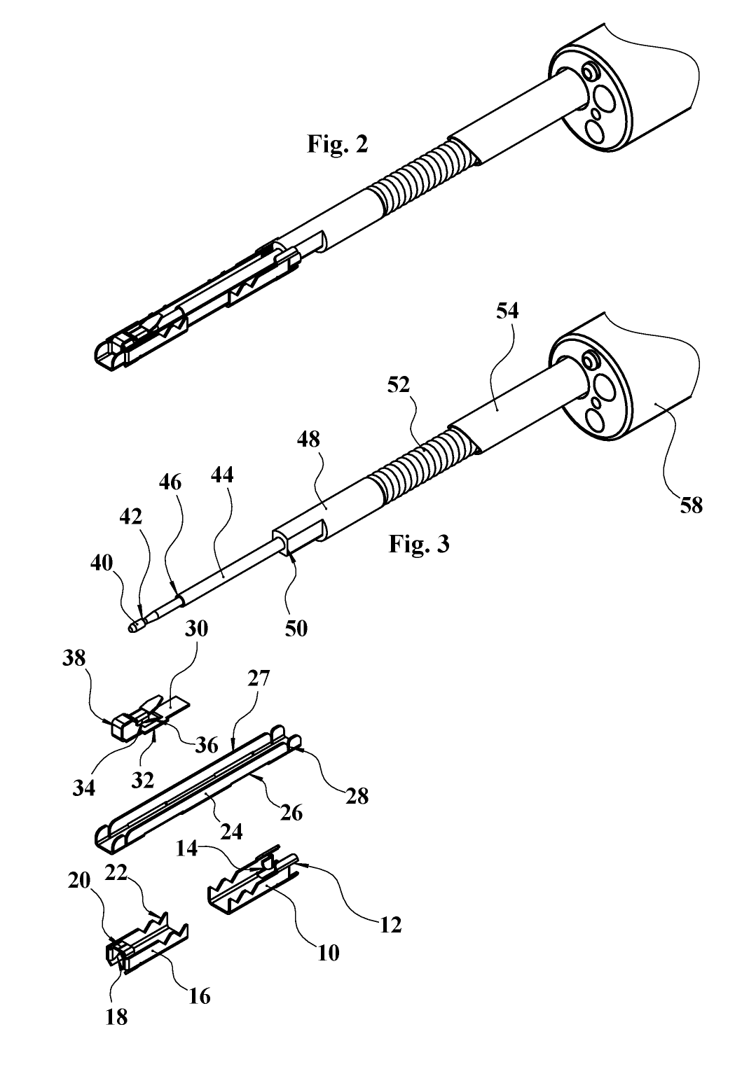

In FIG. 1-1, the sheath 54 is represented by a semi-transparent tube. In FIG. 1-2, the distal prong 16 located at the distal end of the endoscopic clip device is seen emerging from the sheath end 56. As shown in FIG. 1-2, the distal prong 16 deploys as it emerges from the sheath end 56. The substantially perpendicular deployment of distal prong 16 results from the release of tension on the distal prong 16 as its shape is no longer constrained by sheath 54. FIG. 1-3 depicts the emergence of proximal prong 10 as it emerges from sheath end 56. Similar to distal prong 16, proximal prong 10 also deploys as it emerges from the sheath end 56. The substantially perpendicular deployment of proximal prong 10 results from the release of tension on the proximal prong 10 as its shape is no longer constrained by sheath 54. As depicted, each of the distal prong 16 and proximal prong 10 are attached to rail 24. After the distal prong 16 and proximal prong 10 of the endoscopic clip device are each configured in a substantially perpendicular orientation relative to the rail 24 in FIG. 1-3, as shown in FIG. 1-4, the sheath 54 and, specifically, the sheath end 56 is used to push the proximal prong 10 towards the distal prong 16; meanwhile the rail 24 is held in a substantially fixed position relative to proximal prong 10. As shown in FIG. 1-5, the distal prong 16 and the proximal prong 10 have been brought close together along railing 24 in a closed or clamped configuration, and the distal prong 16, proximal pong 10, and railing component 24 have been released in the closed or clamped configuration and the sheath 54, together with all of the other endoscopic clip device components, has been removed, leaving the clip in place.

FIG. 2 shows an isometric view of the distal end of the endoscopic clip system protruding from the distal end of a generic endoscope 58 shown for reference. The sheath 54, which holds the prongs in a flush non-deployed position, has been cut back for clarity.

FIG. 3 shows an exploded view of FIG. 2. As depicted the endoscopic clip system is seen emerging from a passage provided in a sample endoscope 58. The endoscopic clip system is generally arranged in concentric or nested layers, wherein the outermost layer is the sheath 54, followed by the next layer comprising the flex shaft 52 and concentrically aligned coupler 48, which is followed by the next layer comprising the tube 44 which may telescope at the coupler end 50 within the flex shaft 52 and concentrically aligned coupler 48, which is followed by the next layer comprising the latch wire 40. The latch wire 40 telescopes within tube 44 and is shown emerging from tube end 46. Latch ledge 42 on latch wire 40 is used to hold the clip device in place during deployment and is shaped to provide mechanical release of the deployed clip device upon application of pressure from the tube end 46 of telescoping tube 44. Specifically, the shaped latch ledge 42 near the end of latch wire 40 is shown as a tapered region separating the distal head end of latch wire 40 and the remaining portion of the latch wire 40.

The endoscopic clip device includes a latch plate 30 that is snapped into, perhaps by friction fit, rail 24. The latch plate 30 functions to releasably hold latch wire 40 during clip deployment via latch ledge 42. The latch plate 30 includes latch slide tabs 32 along the lateral sides of the bottom of the latch plate 30, latch guide tabs 34 along the lateral sides of the top of latch plate 30, and a latch hook 36 at a distal end of latch plate 30. Also included with latch plate 30 is a latch flex region 38 which, here is depicted as a “U-shaped” region near the back of the latch plate 30. Release of latch wire 40 from latch plate 30 is accomplished by application of pressure on latch flex region 38 by tube end 46 of tube 40, wherein the pressure on the latch flex region 38 forces the flexible flanges of the latch flex region further apart such that the hold on the latch ledge 42 of latch wire 40 is released and latch wire 40 may be retracted within the tube 40.

The endoscopic clip device includes a three-sided rail 24 at its distal end during deployment that is released once the clip is deployed. Latch slots 26 are located along lateral sides of the rail 24 at the juncture where the floor of rail 24 turns about 90° to form rail sidewalls 27. Also included as part of rail 24 are prong slots 28 which, here are depicted in four locations near either end of rail 24. Rail 24 is sized and configured to fit within the interior cavities defined by proximal prong 10 and distal prong 16.

The endoscopic clip device also includes proximal prong 10. Proximal prong 10 is specially adapted for movable connection to rail 24 in multiple configurations to allow for both rotational movement along a single fixed plane and slidable movement along the horizontal plane defined by the rail 24. Specifically, proximal prong 10 includes push tabs 12 on either top lateral side of proximal prong 10 to provide a surface for application of pressure by the sheath end 56. Also included on proximal prong 10 are slide tabs 14, located at the top end of proximal prong 10 and along either lateral side. Slide tabs 14 are specially shaped to provide a hooked and curved surface. Slide tabs 14 directly and movably interact with prong slots 28 located on rail 24. The curved or rounded surface of slide tabs 14 permits one-way rotational movement of the proximal prong 10. That is, as proximal prong 10 deploys, the slide tabs 14 rotate out of slots 28 and about a hooked connection to rail 24, and the rotational movement is permitted by the curved surface interaction between the slide tabs 14 of proximal prong 10 and the slots 28 of rail 24, which also include a matching curved surface to accommodate this rotational movement. Proximal prong 10 is depicted with multiple teeth 22, but such teeth may, optionally, not be included, or be included in other numbers or configurations.

Distal prong 16 is specially adapted for movable connection to rail 24 in a rotational movement along a single fixed plane but, unlike proximal prong 10, does not allow for slidable movement of the distal prong 16 along the horizontal plane defined by the rail 24. That is, distal prong 16 is located at the distal end of rail 24. Slide tabs 18 directly and movably interact with prong slots 28 located on rail 24. The curved or rounded surface of slide tabs 18 permits one-way rotational movement of the distal prong 16. That is, as distal prong 16 deploys, the slide tabs 18 rotate out of slots 28 and about a hooked connection to rail 24, and the rotational movement is permitted by the curved surface interaction between the slide tabs 18 of distal prong 16 and the slots 28 of rail 24, which also include a matching curved surface to accommodate this rotational movement. Distal prong 16 includes bridge 20 which provides structural reinforcement and also upon deployment moves into a fixed or locked connection with rail 24. Similar to proximal prong 10, distal prong 16 is depicted with multiple teeth 22, but such teeth may, optionally, not be included, or be included in other numbers or configurations.

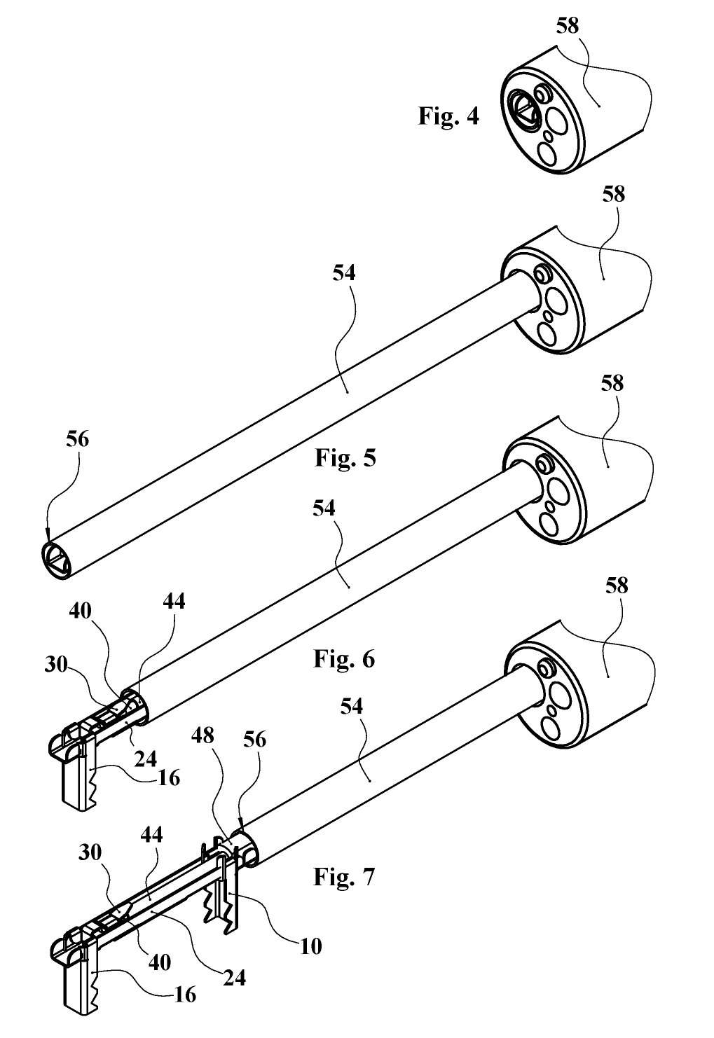

FIGS. 4 through 14 depict a complete sequence of deployment of the endoscopic clip systems and release of the endoscopic clip.

FIG. 4 shows an isometric view of the distal end of the endoscope 58 with the distal end of the endoscopic clip system roughly flush with the end of the endoscope.

FIG. 5 shows an isometric view of the distal end of the endoscope 58 with the distal end of the endoscopic clip system protruding from the distal end of the endoscope 58 and the sheath 54, with sheath end 56, fully covering the distal end of the endoscopic clip system.

FIG. 6 shows an isometric view of the distal end of the endoscope 58 with the distal end of the endoscopic clip system partially deployed as it protrudes from the distal end of the endoscope with the sheath 54 fully exposing the distal prong 16, allowing said prong to rotate in to a substantially perpendicular position. Specifically, rail 24 is shown partially exposed. Also shown are latch plate 30, latch wire 40 and tube 44.

FIG. 7 shows an isometric view of the distal end of the endoscope 58 with the distal end of the endoscopic clip system fully deployed as it protrudes from the distal end of the endoscope with the sheath 54 fully exposing both the distal prong 16 and proximal prong 10 allowing both said prongs to rotate. Also shown are rail 24, latch plate 30, tube 44, coupler 48, and sheath end 56.

FIG. 8 shows an isometric view of the distal end of the endoscope 58 with the distal end of the endoscopic clip system protruding from the distal end of the endoscope with the end 56 of the sheath 54 pushed up against the back of the proximal prong 10, and push tabs 12. Here, coupler 48 is not visible. Depending on the tissue site to be addressed, the endoscopic clip of the present invention could be locked into this maximally open position to address larger tissue site closures.

FIG. 9 shows an isometric view of the distal end of the endoscope 58 with the distal end of the endoscopic clip system protruding from the distal end of the endoscope with the end 56 of the sheath 54 pushed up against the back of the proximal prong 10, and push tabs 12, causing said prong to travel roughly half the distance down the rail 24. Depending on the tissue site to be addressed, the endoscopic clip of the present invention could be locked into this partially open position.

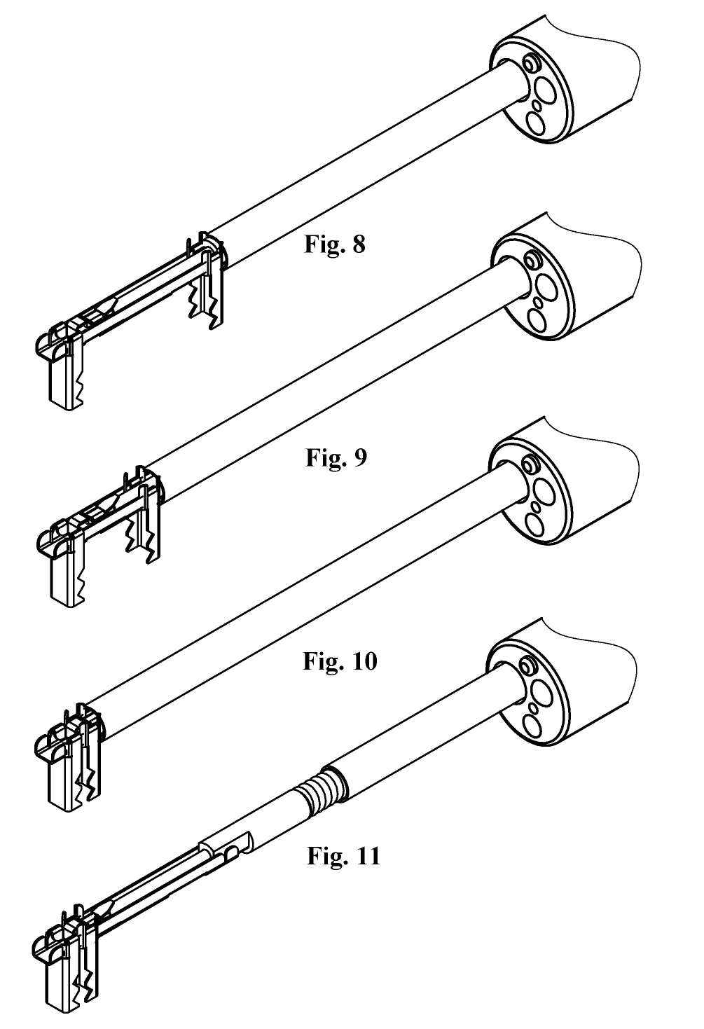

FIG. 10 shows an isometric view of the distal end of the endoscope 58 with the distal end of the endoscopic clip system protruding from the distal end of the endoscope with the end 56 of the sheath 54 pushed up against the back of the proximal prong 10, and push tabs 12, causing said prong to travel completely down the rail 24 for a maximally closed or clamped position as the distal clip 16 and proximal clip 10 are brought together. In this embodiment, distal clip 16 may have a surface that is adjacent to and in direct contact with a surface of the proximal clip 10.

FIG. 11 shows an isometric view of the distal end of the endoscope 58 with the distal end of the endoscopic clip system protruding from the distal end of the endoscope with the end 56 of the sheath 54 pulled back to expose the entire rail 24 in order to prepare for release.

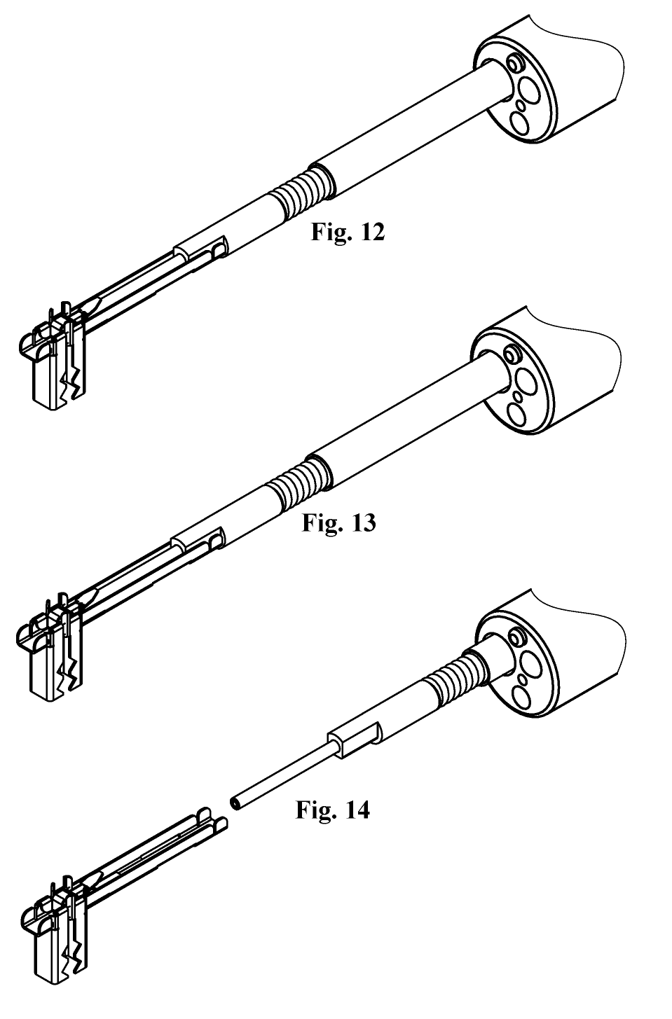

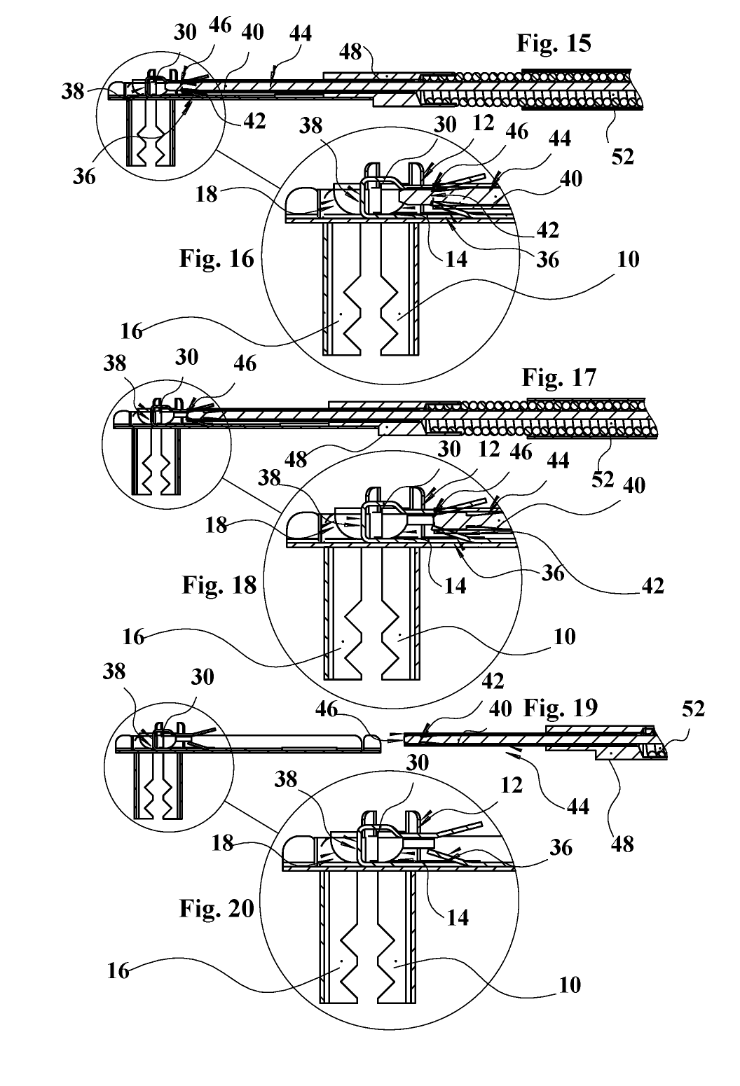

FIG. 12 shows an isometric view of the distal end of the endoscope 58 with the distal end of the endoscopic clip system protruding from the distal end of the endoscope with the end 56 of the sheath 54 pulled back to expose the entire rail 24 while the coupler 48 is fully engaged with the rail 24. This view also represents the release of the latch wire 40 from the latch plate 30; however, the latch wire 40 release is obscured in this view (but is visible in FIGS. 15 and 16). FIG. 15 shows a cross sectional view of FIG. 12 and FIG. 16 shows a detail view of the distal end in FIG. 15.

FIG. 13 is substantially similar to FIG. 12 and shows an isometric view of the distal end of the endoscope 58 with the distal end of the endoscopic clip system protruding from the distal end of the endoscope with the end 56 of the sheath 54 pulled back to expose the entire rail 24. This figure represents the retracting of the latch wire 40 into the tube 44. This is obscured in this view, but is visible in FIGS. 17 and 18. FIG. 17 shows a cross sectional view of FIG. 13 and FIG. 18 shows a detail view of the distal end in FIG. 17.

FIG. 14 shows the distal end of the endoscopic clip system as released and pulled away from the remaining components endoscopic clip assembly. As shown, the entire rail, the latch plate, distal clip 16, and proximal clip 10, are clipped or clamped at a tissue site, then released and left in place. It is contemplated that rail length for endoscopic clips of the present device may be selected to match expected tissue site closure sized in order to avoid leaving excess rail protruding from applied clips at treated tissue sites. FIG. 19 shows a cross sectional view of FIG. 14 and FIG. 20 shows a detail view of the distal end in FIG. 19.

FIGS. 15 through 20 provide sequential cross sectional and detail views of the deployment and release mechanisms. Please see the description provided in connection with FIG. 3 in connection with each of the reference identifiers provided in FIGS. 15 through 20, and those descriptions associated with each of FIGS. 12, 13, and 14 as noted above in associated with each of FIGS. 15 through 20, respectively.

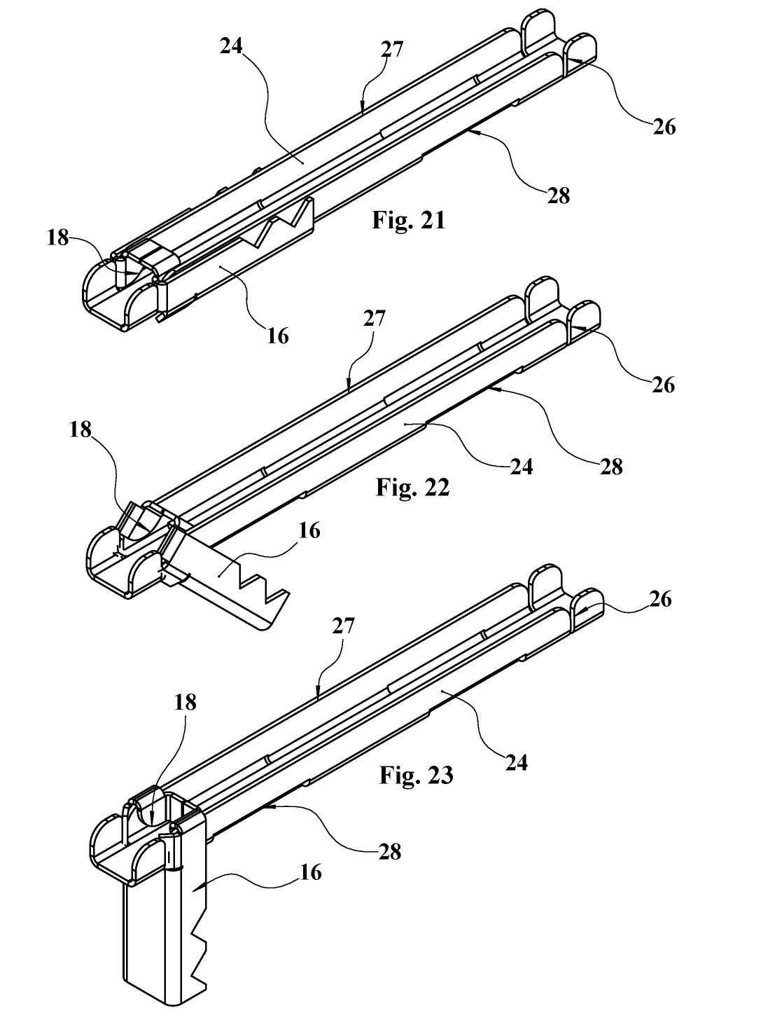

FIGS. 21 through 23 provide isometric views of the rail 24 and distal prong 16 prior to and during deployment and as fully deployed and locked into position. Please see the description provided in connection with FIG. 3 in connection with each of the reference identifiers provided in FIGS. 21 through 23. FIG. 21 shows an isometric view of the rail 24 and the distal prong 16 in a fully stowed, collapsed, or non-deployed position. FIG. 22 shows an isometric view of the rail 24 and the distal prong 16 in a partially deployed position, wherein the distal prong 16 is not yet in a locked position. FIG. 23 shows an isometric view of the rail 24 and the distal clip 16 in a fully deployed position, wherein the distal clip 16 is in a substantially perpendicular position and is locked into place. Lateral and vertical movement of the fully deployed distal prong 16 is substantially prevented due to the close fitting nature of the rail 24 and distal prong 16 component parts. Additionally, the bridge 20 of the distal prong 16 also acts to limit movement as it has a surface located adjacent to the interior cavity of rail 24.

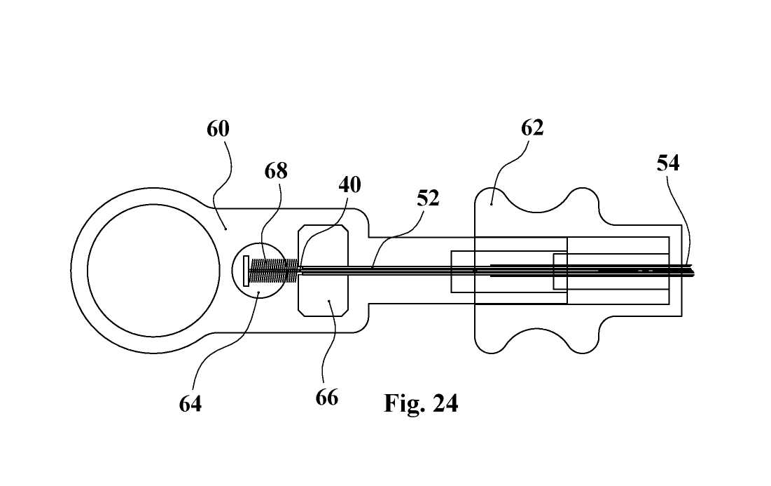

FIG. 24 shows a side view of the proximal control end of the endoscopic clip system. Here, the latch wire 40 is shown as associated with both tension spring 68 and release button 64. It is noted that the tension imposed by the tension spring 68 and through the latch wire 40 provides the force used to deploy the distal prong 16 and the proximal prong 10 of the endoscopic clip device. While tension spring 68 is depicted here, other components may be used or substituted for tension spring 68 such as, for example, electric force components, etc. Flex shaft 52 is depicted here as being substantially co-terminus with the latch wire 40 upon junction of the latch wire 40 with the tension spring 68. In this configuration, release button 64 may be manually compressed to increase tension on the latch wire 40; meanwhile, release of the latch wire 40 from the latch plate 30 at the distal end of the endoscopic clip device is accomplished by separate application of pressure from the tube end 46 against the latch flex region 38, the flanges of which then spread apart releasing their hold on the latch ledge 42. The released latch wire 40 may then be retracted through the tube 44. Upon disassociation of the latch wire 40 with the latch plate 30, the rail 24, latch plate 30, distal prong 16, and proximal prong 10 are released from the remaining endoscopic clip device components and left in place at a treated tissue site.

Also shown in FIG. 24 is rotational wheel 66 which is located on the exterior of flex shaft 52 to allow for rotational manipulation of the endoscopic clip device. Fixed handle 62 is also depicted and connects with sheath 54. Moving handle 60 provides for manipulation of all the components of the endoscopic clip device except for the sheath which is controlled by manipulation of fixed handle 62.

In another alternative embodiment of the present invention, the clip rail and prong configuration, the inventors also contemplate use of a “W-shaped” or “M-shaped” clip, which is intrinsically biased to open and easily slide through a delivery cartridge similar to the existing concepts. Thus, as this biased clip is exposed from the end of the tube, it regains its desired opposing jaw shape. A backbone or substrate may penetrate each fold of the clip, such that as it is tightened on tissue, the clamp is prevented from opening. This “W-shaped” or “M-shaped” clip may then be cut free or released from the cartridge.

Other embodiments of the present invention include use of the clip could potentially be used to carry a payload of therapeutic material. For example, a small “pad” or “strip” impregnated with some substance may be fitted into or on the tissue facing surfaces or within the interior cleft of the prongs. In yet another embodiment, a payload of therapeutic material may be worked into the rail.

In other alternative embodiment the endoscopic clip of the present invention is used as an anchor to secure an object (catheter, location device, etc.) to the target tissue site, for example, to mucosal tissue.

It is to be understood that while the invention has been described in conjunction with the detailed description of the embodiments depicted in the Figures, the foregoing description is intended to illustrate and not limit the scope of the invention, which is defined by the scope of the appended claims. Other aspects, advantages, and modifications are within the scope of the following claims.

1. An endoscopic clip configured relative to a medical device comprising a rail, a distal prong, and a proximal prong, wherein the distal prong and the proximal prong are subject to tension delivered by a latch wire.

2. The endoscopic clip of claim 1, wherein the distal prong and the proximal prong are each movably connected to the rail.

3. The endoscopic clip of claim 2, wherein the distal prong and the proximal prong are configured for sequential deployment along spaced apart rail locations.

4. The endoscopic clip of claim 1, wherein the proximal prong is slidable on the rail.

5. The endoscopic clip of claim 1, wherein at least one of the distal prong and the proximal prong is rotatable.

6. The endoscopic clip of claim 1, wherein at least one of the distal prong and the proximal prong includes a slide tab comprising a protrusion, lip, rounded or curved surface.

7. The endoscopic clip of claim 1, wherein at least one of the distal prong and the proximal prong, when in a deployed configuration, is substantially perpendicular to the rail.

8. The endoscopic clip of claim 1, further comprising a-latch wire release mechanism.

9. The endoscopic clip of claim 8, wherein the latch wire release mechanism comprises a releasable snap fit within a latch plate connected to the rail.

10. The endoscopic clip of claim 1, further comprising a medical device and wherein the endoscopic clip is rotatable along a longitudinal axis of the medical device.

11. An endoscopic clip configured relative to a medical device comprising a folding distal prong and a folding proximal prong, wherein the distal prong and the proximal prong are subject to tension delivered by a latch wire.

12. The endoscopic clip of claim 11, further comprising a releasable rail connected to each of the folding distal prong and the folding proximal prong.

13. The endoscopic clip of claim 12, wherein the folding distal prong and the folding proximal prong are locked in a substantially perpendicular configuration relative to the rail.

14. The endoscopic clip of claim 11, further comprising a medical device and wherein the endoscopic clip is rotatable along a longitudinal axis of the medical device.

15. A method of using an endoscopic clip configured relative to a medical device, wherein the endoscopic clip comprises a rail, a distal prong, and a proximal prong, and wherein the distal prong and the proximal prong are subject to tension delivered by a latch wire and comprising sequentially deploying the distal prong and the proximal prong.

16. The method of claim 15, comprising applying pressure to a sheath end of the medical device to move the proximal prong towards the distal prong.

17. The method of claim 15, wherein the deployed endoscopic clip is used to stop gastrointestinal bleeding, mark locations, or close perforations and mucosal defects.

18. The method of claim 15, comprising introducing the endoscopic clip into a sheath in a folded configuration and retracting the sheath to provide the endoscopic clip in an unfolded configuration immediately parallel to a tissue treatment site.

19. The method of claim 15, further comprising providing a medical device and rotating the endoscopic clip along a longitudinal axis of the medical device.

20. The method of claim 15, further comprising applying pressure to a latch flex region with a tube end.
